(19) United States
(12) Patent Application Publication
(10) Pub. No.: US 2019/0160213 A1
(43) Pub. Date: May 30, 2019

(54) APPARATUS, METHODS AND SYSTEMS FOR DYNAMIC VENTRICULAR ASSISTANCE

(71) Applicant: Richard Wampler, Loomis, CA (US)

(72) Inventor: Richard Wampler, Loomis, CA (US)

(21) Appl. No.: 15/826,663

(22) Filed: Nov. 29, 2017

(51) Int. Cl. A61M 1/10; A61M 1/12; A61B 5/024

(52) U.S. Cl. CPC A61M 1/1086; A61B 5/024; A61M 1/122

(57) ABSTRACT

Systems methods are disclosed for changing one or more characteristics (e.g. flow magnitude via pump speed) of mechanical circulatory assistance provided by an LVAD during specified points in the cardiac cycle, preferably using closed loop control. The system and method may be implemented for dynamically changing ventricular unloading during the cardiac cycle by adjusting the degree of ventricular assistance during systole and/or diastole. The system and methods also include a means to sense the phase of the cardiac cycle to inform the LVAD of timing within the cardiac cycle.

### CROSS-REFERENCE TO RELATED APPLICATIONS

Not Applicable

### STATEMENT REGARDING FEDERALLY SPONSORED RESEARCH OR DEVELOPMENT

Not Applicable

### NOTICE OF MATERIAL SUBJECT TO COPYRIGHT PROTECTION

A portion of the material in this patent document may be subject to copyright protection under the copyright laws of the United States and of other countries. The owner of the copyright rights has no objection to the facsimile reproduction by anyone of the patent document or the patent disclosure, as it appears in the United States Patent and Trademark Office publicly available file or records, but otherwise reserves all copyright rights whatsoever. The copyright owner does not hereby waive any of its rights to have this patent document maintained in secrecy, including without limitation its rights pursuant to 37 C.F.R. § 1.14.

### BACKGROUND

### 1. Technical Field

The technology of this disclosure pertains generally to cardiac devices, and more particularly to mechanical circulatory support devices.

### 2. Background Discussion

Congestive heart failure (CHF) is a major, rapidly growing public health problem, with a prevalence of more than 5.8 million in the United States and more than 23 million worldwide that results in hundreds of thousands of deaths annually. After the diagnosis of CHF, survival estimates are 50% and 10% at 5 and 10 years, respectively, and left ventricular dysfunction is associated with an increase in the risk of sudden death. Patients with end stage heart failure who are refractory to medications, surgical intervention and resynchronizer pacing are best treated with cardiac transplantation. However, donor hearts are limited to about 2,000 per year in the United States and, consequently, there is a large unmet need for approximately 75,000 patients who would benefit from cardiac transplantation, but for whom no donor heart is available.

Extensive research since the mid 1960's has resulted in numerous surgically implanted left ventricular assist devices (LVAD) intended to take over part or all of the work of the left ventricle while working in parallel with the native heart. First generation LVAD's were based on biomimicry and emulated the pulse using positive displacement pumps. Although these technologies demonstrated potential clinical benefit, they had limited durability. The current generation of LVAD's is based on rotary pump technologies and has proven to be effective, durable, and result in significant reduction in pulsatility. Rotary pumps impart kinetic energy to a fluid, as velocity, by means of rotating blades to provide continuous flow. The velocity energy of the fluid is then converted to static pressure to move blood through the circulation. A major advantage of rotary LVADs is superior durability, sometimes running for over 10 years.

The three commercially available rotary LVAD's in clinical use in the United States are, the HeartMate II and HeartMate III owned by Abbott, Inc. and the HeartWare HVAD owned by Medtronic, Inc. These devices have demonstrated the ability to support systemic circulation and have been effective in bridging patients to cardiac transplantation. In addition, these devices have demonstrated the ability to support patients who are not candidates for transplantation for as long as 10 years, so called ‘destination’ patients. However, expectations that patients would recover during LVAD support and, ultimately, be weaned from support have not been realized.

The poor weaning results (<5%) are not understood. Failure to achieve sufficient myocardial recovery to permit weaning may be due to the fact that the myocardium is irreversibly damaged. Alternately, it may be that the limitations of existing pump control do not lend itself to optimally conditioning the myocardium.

Although existing LVAD's provide greatly improved circulatory support for patients in heart failure, and provide successful bridging to transplantation and long term support, they are very limited in their ability to meet the dynamic demands of the heart and circulation. Presently, they operate only under speed control, meaning the motor strives to maintain speed regardless of the load using a closed loop algorithm. If there is a clinical reason to vary speed, the only control available to the clinician is to manually adjust the speed of the pump during clinic visits. The speed cannot be changed remotely by the physician, nor can the patient adjust the speed. In fact, there is no intelligence in existing LVADs to automatically adjust to changing clinical conditions of patients. For this reason the pumps are ‘dumb’ in that the degree of assistance is determined only by the pressure gradient against which it is pumping. Consequently, most of the flow occurs during systole, when the pressure difference between the inlet and outlet is low, and significantly less during diastole, when the pressure difference is greater. This behavior of the LVAD is driven by the pressure difference imposed by the ventricle across the pump inlet to outlet whether or not the ventricle might benefit from a different strategy.

Given the limitations of existing rotary LVADs, only the speed can be adjusted to encourage myocardial recovery. The present weaning strategy, typically, lowers the pump speed based on a ‘ramp test’ with the intention of increasing the work load of the heart for a period of months. Ramp testing provides an assessment of ventricular performance by measuring the dilation of the heart in response to trials of decreased pump speed. Some patients tolerate decreased assistance and may well become weanable, but they also may be in a state of increased heart failure compared to their clinical state at a previous higher speed.

Accordingly, an objective of the present disclosure is a system and method for LVAD treatment to affect myocardial recovery and thus make it possible to remove the device or increase the number of patients weaned from support. With an increase in weaning rates the need for cardiac transplantation may be decreased, and mechanical circulatory assistance may be employed as a definitive treatment, rather than bridging to transplantation or palliative care. In addition, LVADs could be used on less sick patients as a therapy to recovery and weaning rather than transplantation.

### BRIEF SUMMARY

An aspect of the present disclosure is a system and method for changing one or more characteristics (e.g. flow magnitude via pump speed) of mechanical circulatory assistance provided by a left ventricular assist device (LVAD) during the cardiac cycle using closed loop control.

Another aspect is a system and method for dynamically changing ventricular unloading during the cardiac cycle in patients who are recipients of left ventricular assist devices LVADs. The system and methods provide for a platform to adjust the degree of ventricular assistance, independently, during systole and/or diastole. The system and methods also provide for a means to sense the phase of the cardiac cycle to inform the LVAD of timing within the cardiac cycle.

One method for dynamic ventricular unloading (DVU) in accordance with the present description is the optimization of mechanical ventricular assistance to affect a higher rate of heart recovery and increase the ability of clinicians to wean patients from LVAD support.

Another method for dynamic ventricular unloading in accordance with the present description is the reduction of the residence time of the blood in the ventricle to minimize thrombosis and strokes.

Further aspects of the technology described herein will be brought out in the following portions of the specification, wherein the detailed description is for the purpose of fully disclosing preferred embodiments of the technology without placing limitations thereon.

### BRIEF DESCRIPTION OF THE SEVERAL VIEWS OF THE DRAWING(S)

The technology described herein will be more fully understood by reference to the following drawings which are for illustrative purposes only:

FIG. 1 shows a high-level schematic diagram of a system for providing mechanical circulatory assistance provided by a left ventricular assist device (LVAD) during the cardiac cycle.

FIG. 2A and FIG. 2B show graphs of flow pulsatility, before (FIG. 2A) and after (FIG. 2B) manipulation via Dynamic Ventricular Unloading (DVU) in accordance with the present description.

FIG. 3 shows a schematic flow diagram for a method for providing dynamic ventricular unloading via an LVAD pump in accordance with the present description.

FIG. 4 illustrates a graph showing the hydraulic performance (flow vs. head pressure) for a typical LVAD pump at varying speeds.

FIG. 5 shows an exemplary user interface for operating an LVAD pump with dynamic ventricular unloading.

### DETAILED DESCRIPTION

The present description is directed to controlling the distribution of assistance during the cardiac cycle for improved heart muscle function, and in particular optimize the degree of ventricular unloading generated by a left ventricular assist device (LVAD) device during the acute phase of treatment. Once the condition has stabilized, precise control of reloading of the heart may be applied to increase the strength of ventricular contraction, optimally leading to the removal of the LVAD device from the patient. Removal of the device would permit the patient to return to a more normal life style. Weaning is also desirable because risk of significant adverse events, including severe stroke, increases with time, thus the incidence of adverse events would be expected to decrease.

In one embodiment, the cardiac muscle is conditioned by timing operation of the LVAD to ‘work out’ the heart with short intervals of increased exertion followed by periods of rest, similar to strategies used to condition skeletal muscle. Such an exercise program, administered over a period of (e.g. months), would ‘work out’ the heart according to a scheduled regimen of gradually increasing exertion with rest. The systems and method of the present invention would ideally provide an LVAD with the ability to adjust the level of assistance during the cardiac cycle to precisely control the work load of the heart from beat to beat (e.g. intra-beat manipulation) and would have the ability to gradually and precisely decrease or increase ventricular support as desired. As recovery progresses, the exercise regimen may be adjusted to greater exertion to maximize conditioning and permit removal of the LVAD.

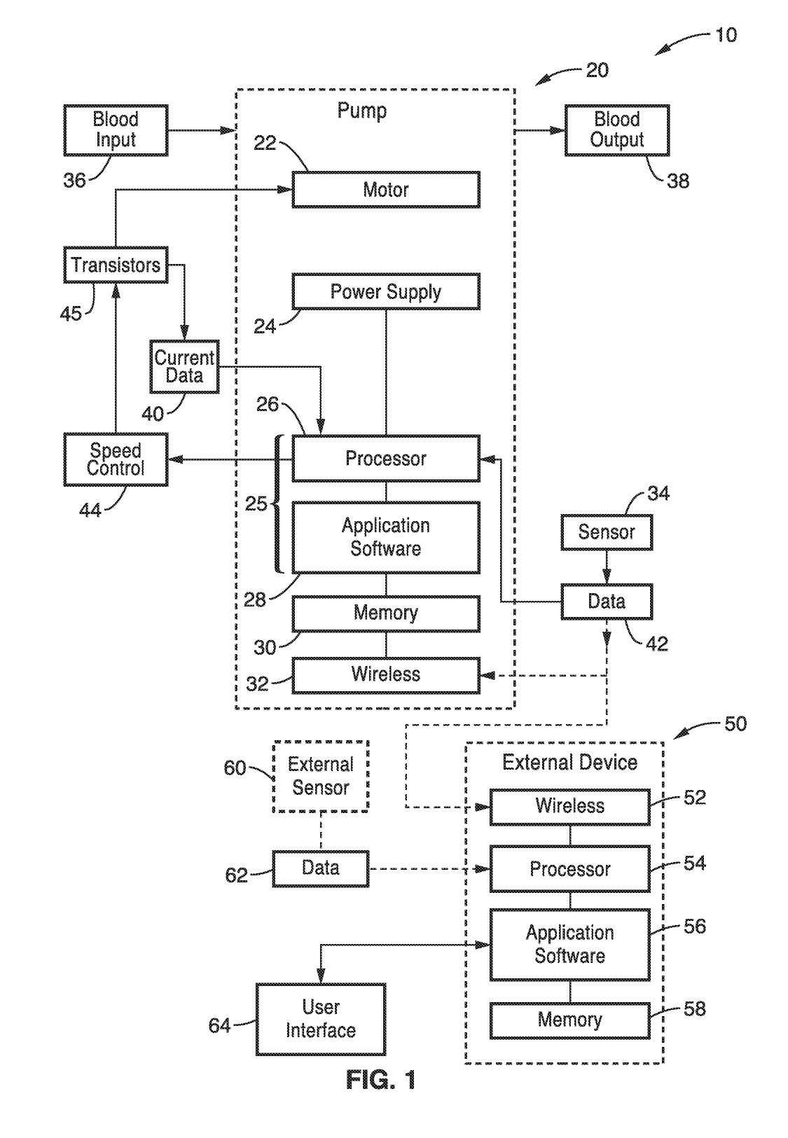

FIG. 1 shows a high-level schematic diagram of a system 10 for providing mechanical circulatory assistance provided by an LVAD pump 20 during the cardiac cycle. The LVAD pump 20 is disposed within the circulatory system so as to receive blood at input 36 and provide circulatory assistance via blood output 38. LVAD pump 20 generally comprises a motor 22, power supply 24 (e.g. battery of like power source) supplying voltage to the motor 22, and programming and circuitry to control operation of the motor 22, such as application software 28 stored in memory 30 for execution on processor 26. Application software 28 generally comprises instructions executable on processor 26, which together operate as controller 25 commutate the motor 22 and directly supply driving power to the motor 22, in addition to determining, maintaining, and changing the motor speed via speed control commands 44. Where direct wiring is not used, pump 20 may also comprise wireless circuitry 32 (e.g. Wi-Fi, Bluetooth, or like wireless communication protocol) for communicating with one or more sensors 34 and one or more external devices 50. In an alternative embodiment, the sensor 34 and/or external device 50 may be hard-wired to the pump 20.

Alternatively, or in addition to data 42 acquired from sensor 34, current measurement data 40 may also be acquired relating to the current provided to the motor 22 (e.g. via power supply 24), and used as a source of feedback to determine flow rate and timing of the heart cycle, as will be explained in further detail below. In typical rotary pumps, very low impedance resistors/transistors 45 are in series with power and commands 44 being delivered to the motor 22 from the controller 25. The voltage drop across the reference resistors 45 may be measured, and then current may be calculated on basis of equation IR=V. Current data 40, and/or sensor data 42 may be used to determine the timing of the cardiac cycle, or other physiological characteristics, to adjust the speed (via a speed control command 44) of the motor during differing stages of the cardiac cycle.

In one embodiment, external device 50 may comprise a cell phone, computer, wearable controller, or other processing device that has application software 56 for communicating with and/or controlling the pump 20. The external device 50 preferably comprises a processor 54 for executing application software 56 that is stored in memory 58, and optional wireless circuitry 52 for communicating with the pump 20 if direct wiring is not used. An external sensor 60 may also be provided for acquiring physiological data 62 from the patient, the data 62 being in addition to, or replacement of data 42 from sensor 34. External device application software 56 may also include a user interface 64 for displaying data and/or control functionality with respect to sensors 60/34 and pump 20.

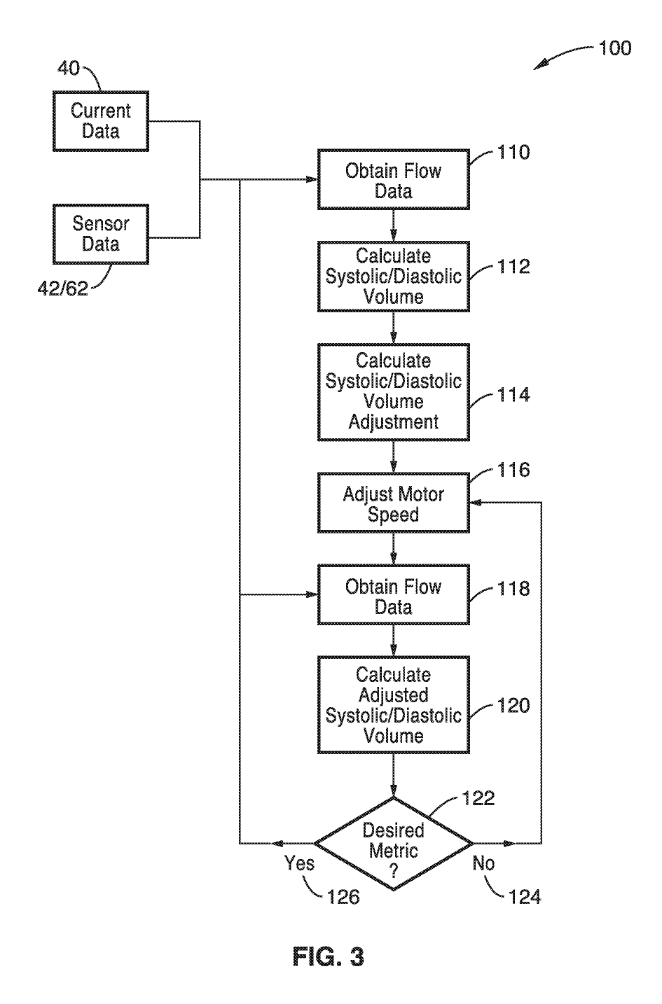

As will be explained in further detail below, one or more of the pump application software 28 and external device application software 56 may include instructions for controlling the pump 20 via a method 100 for dynamic ventricular unloading (DVU) via continuous feedback provided by input from one or more of motor current data 40 and sensor data 42/62, as shown in FIG. 3 and provided in further detail below.

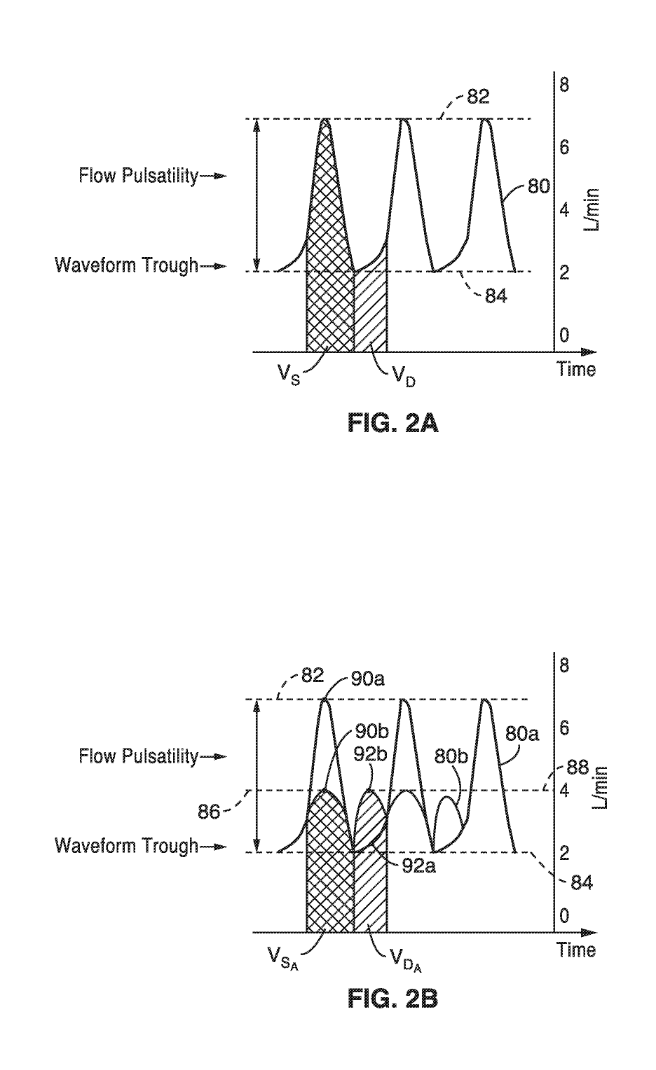

FIG. 2A and FIG. 2B show graphs of the instantaneous pump output flow over time, i.e. pulsatility curve 80, before (FIG. 2A) and after (FIG. 2B) manipulation via Dynamic Ventricular Unloading (DVU) in accordance with the present description. The pulsatility curve 80 in the cardiac cycle will have a systolic phase (and corresponding systolic volume Vₛ) that includes an increasing flow through the peak flow 82 that then generally decreases to the waveform trough 84, and a diastolic phase (and corresponding diastolic volume V_{D}) that starts at the trough 84 and extends until the systolic phase. Dynamic ventricular unloading (DVU), in accordance with the methods disclosed herein, is performed by actively changing the degree of unloading during the cardiac cycle rather than unloading based exclusively on the pressure gradient across the LVAD pump 20, thus allowing for the adjustment the ratio of volume occurring during diastole/systole simply by changing the pump speed 44 during the cardiac cycle.

For example, increasing pump speed 44 and corresponding blood output flow 38 (FIG. 1) during diastole increases diastolic volume output by the pump 20 to create an adjusted pumped diastolic volume V_{DA}, as shown in FIG. 2B. It should be noted that diastolic volume V_{DA} is the volume generated by the pump, and is not to be confused with end diastolic volume (EDV), which is generally understood to mean the volume of blood in the right and/or left ventricle at the end load or filling in diastole. This adjusted diastolic volume V_{DA} has the anatomical affect of a smaller end diastolic volume and a diminished starling response of the ventricle. In addition, the smaller end diastolic volume results in a smaller ventricular wall radius and reduces the wall tension needed to maintain a given pressure. The above condition would maximally unload the ventricle by reducing myocardial oxygen requirements and produce a significant diastolic augmentation that will increase perfusion of the recovering myocardium.

In addition (or alternatively) to the above, output 38 during systole could be decreased by decreasing pump speed 44 to decrease the systolic volume (see adjusted systolic volume V_{SA} in FIG. 2B). This would affect a decrease in pressure in the aorta and decrease in after load on the ventricle to facilitate recovery during the initial phase following LVAD implantation.

After the acute phase of recovery, it may be desirable to evaluate the status of the ventricle and attempt to wean from the patient from LVAD support by decreasing the ratio of assistance during diastole over systole, thus requiring the heart to perform more work. To increase ventricular work, the pump speed 44 and flow 38 would be decreased during diastole, which would result in an increase in the end diastolic volume. Such a maneuver would increase the starling response and increase the wall radius, thus requiring more wall tension and more myocardial work. Flow 38 during systole could also be increased to increase pressure in the aorta and increase after load on the ventricle. The above would also allow for a physician to run a ramp test while observing the behavior of the heart as the ratio of diastolic and systolic flow was varied, rather than just changing the average pump speed. While the above scenarios may actually be better described as Dynamic Ventricular Reloading (DVR), it is appreciated that DVU may be used interchangeably for all Dynamic Ventricular Assistance (DVA) scenarios.

FIG. 3 shows a schematic flow diagram for a method 100 for providing dynamic ventricular unloading via an LVAD pump 20 in accordance with the present description. Method 100 may be carried out as instructions included in one more of application software 28 of pump 20 and application software 56 of external device 50.

Method 100 assumes an LVAD pump configured to gain access to the circulatory system of the patient such that blood can be removed from the left ventricle and pressurized into the aorta. In one exemplary method, a pump 20 is surgically implanted such that an inflow cannula (not shown) is inserted into the ventricular cavity to provide input 36 to the pump. The pump output 38 connects to the aorta via an artificial artery. Blood is then removed from the left ventricle via the inflow cannula and pumped into the aorta, thus, directly assisting the left ventricle. It is appreciated that the above pump implementation is provided for exemplary purposed only, and is not intended to be exhaustive of presently available techniques for circulatory assistance. It is appreciated that the systems and methods disclosed herein may be integrated with, or provided as an add on, for any number of circulatory assistance pumps available in the art.

As provided above, dynamic ventricular unloading adjusts the balance of blood flow between diastole and systole by changing the pump speed 44 of the LVAD pump 20 during the cardiac cycle. To accommodate this, synchronization of the pump 20 with the cardiac cycle is performed via closed loop control by acquiring a signal at step 110 that identifies the phase(s) of contraction, e.g. flow data such as the flow pulsatility curve 80, to identify systole and diastole events/phases in the cardiac cycle. A number of signals may be used, including a signal of the motor current data 40, or sensor data 42/62 from one or more internal or external sensors in the form of an ECG leads (electrocardiogram measurements), intraventricular pressure or volume transducers (left side pressure or ventricular volume measurements).

The patient's EKG would provide a reliable and stable signal, but would typically involve implanted leads in the myocardium and additional signal processing. A preferred embodiment would use the motor current 44 as a surrogate to sense timing of the pulse rate, systole and diastole. As explained in further detail below, method 100 may be configured to detect onset of systole and diastole to trigger speed changes. In addition, the current signature from data 44 may be used as a surrogate to measure LVAD flow and could be used to calculate the ratio of flow during diastole and systole, and then seek the desired ratio of flow during diastole versus systole. A ventricular pressure volume loop may also be implemented to further evaluate the effect of the dynamic assistance.

With respect to motor current data 40, generally there is a direct correlation between the current applied to the motor and the blood flow rate. This pump-specific data may be used to determine flow rate, and the corresponding phase of the cardiac cycle, by identifying the change in motor current over time.

This data acquisition is performed in real time, to accommodate for changes in heart rate, and allow for rapid change in speeds during the cardiac cycle, as well provide instructions/control the pump 20 to change speed by a predetermined protocol.

With the acquired data from step 110, the flow pulsatility curve may be generated, and systole and diastole phases identified to calculate systolic volume Vₛ and diastolic volume V_{D} (FIG. 2A) at step 112 by integrating under the curve at respective identified timeframes. The ratio of flow during diastole and systole may then be calculated, e.g. as either V_{D}/Vₛ or V_{D}/(V_{D}+Vₛ), or any desired arithmetic or algebraic formula.

At step 114, a desired systolic/diastolic volume adjustment is calculated based on the pump flow or flow ratio in relation to a target flow or flow ratio. The target flow is preferably based on a treatment protocol that is desired for the patient at that time (e.g. ventricular unloading for acute recovery phase, and ventricular reloading for weaning the patient off the pump).

At step 116, the algorithm then calculates a motor speed adjustment that is predicted to affect the desired systolic/diastolic volume adjustment and corresponding target flow, and sends a command to the motor 22 to change speed accordingly (e.g. increasing motor speed in diastole and/or decreasing or shutting off the motor in systole). The motor speed adjustment may be calculated as a function of a number of factors, including the patient's stroke volume, the blood input flow 36, blood output flow 38, pump speed, pulse rate, etc. The motor speed adjustment is timed with respect to the cardiac cycle based on the pulsatility curve, or other physiological sensor data acquired at step 110. It is appreciated that because of transmission delay, and lag in pump speed manipulation, that timing of the speed adjustment instruction 44 (FIG. 1) may well precede the actual event (e.g. a command to increase speed during diastole may be initiated at some point in systole).

In order to execute DVU, the LVAD pump 20 is preferably configured to be able to change speeds (i.e. slew rate) rapidly within the cardiac cycle. The slew rate of a pump is generally defined as change in rpm/second, and is typically determined by the design of the motor. In a preferred embodiment, a pump having a slew rate of at least 1500 rpm would be desirable.

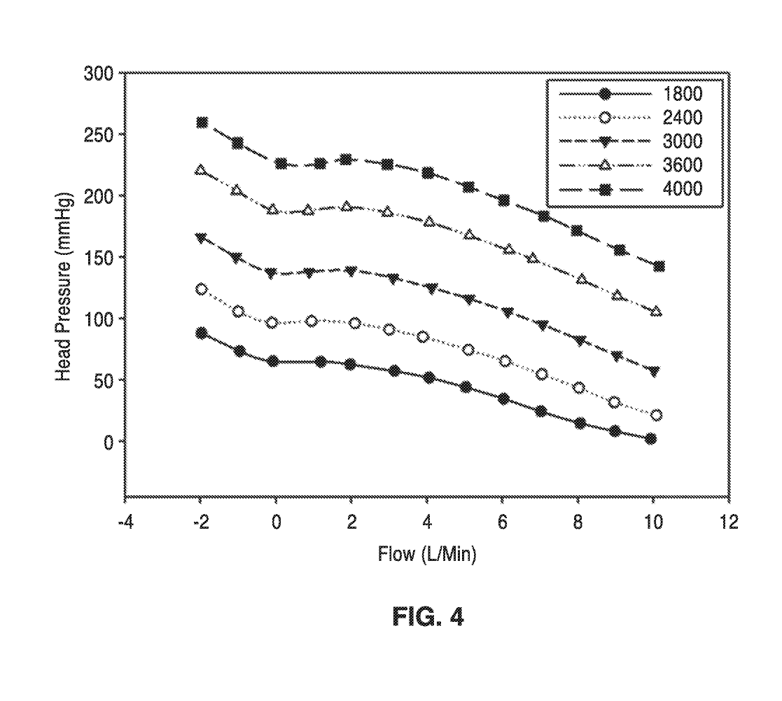

Furthermore, rotary LVAD pumps typically have a ‘slip,’ which means that for a given speed, the flow produced at higher pressure is significantly less than at lower pressures. The hydraulic performance of the HeartWare HVAD™ is shown in FIG. 4. As an example, based on the hydraulic performance in the graph of FIG. 4, the flow at 2,400 rpm is 2 lpm @ 100 mm Hg. Increasing the speed to 3,000 rpm would increase the flow to 6 lpm @ 100 mm Hg. The total time to change speed would be 600 rpm/2000 rpm/second or 300 milliseconds. This implies the ability to dynamically support a heart rate of about 100 bpm. Optimization of the pump 20 may also be implemented increase the slew rate if needed. In a preferred embodiment, the LVAD pump 20 is able to deliver maximum flow rate of at least 5 liters per minute at a pressure of 90 mm Hg.

At step 118, flow data is reacquired via additional input from one or more of current data 40 and sensor data 42/62. The sensitivity of this feedback (e.g. how many heartbeats or seconds between the adjustment and step 116 and reacquisition at step 118) may be adjusted as desired by the physician.

At step 120, the acquired data from step 118 is used to calculate adjusted systolic volume V_{SA} and adjusted diastolic volume V_{DA} (e.g. by regenerating the flow pulsatility curve, and identifying systole and diastole phases as shown in FIG. 2B).

In addition, rotary pumps are capable of creating significant negative pressure at the pump inlet 36, which can result in ‘sucking down’ on the endocardial surface of the heart, effectively blocking the pump inlet 36. As pump speed is increased during diastole with a resultant decrease in VEDV, the risk of ‘suck down’ may be increased. Consequently, ‘suck down’ detection will be necessary to mitigate this undesirable behavior.

At step 122, V_{SA} and V_{DA} or a ratio thereof (e.g. either V_{DA}/V_{SA} or V_{DA}/(V_{DA}+V_{SA}, or other metric) are compared against the target volume metric. If target ratio or other metric is not achieved, another systolic/diastolic volume adjustment is calculated at step 124 to affect another change in motor speed at step 116. Steps 118 through 122 are then repeated in the feedback loop shown in FIG. 3.

It is appreciated that while systolic/diastolic volume ratios are provided as the target metric in the above stems, any number of physiological characteristics (e.g. stroke work index, ejection fraction, etc.) may be used as the target metric, as preferred by the physician.

The query at step 122 may also poll the obtained flow data at 118 for the timing of the adjusted pulsatility curve 80b (contrasted with original curve 80a in FIG. 2B) and readjust the timing of the speed control command 44 if there are any variations necessitating it (e.g. change or offset in heart rate).

If query step 122 reveals that the target ratio or other metric is achieved, than the algorithm continues to poll the sensors to obtain flow data at step 118 at the specified sensitivity increment, and steps 120 through 122 are then repeated in the feedback loop shown in FIG. 3.

As the systolic/diastolic volume ratio is adjusted, conventional clinical metrics can be employed to assess the patient's status. This could include non-invasive modalities such as the EKG, blood pressure, pulse oximetry etc. Invasive tests could include placement of a Swan Gantz catheter for measurement of mixed venous saturation, right sided pressures and cardiac output. During the weeks to months after DVU adjustment the response of the myocardium can be assessed by echocardiography and various blood markers that measure myocardial injury (enzymes) and heart failure (BPN, troponin).

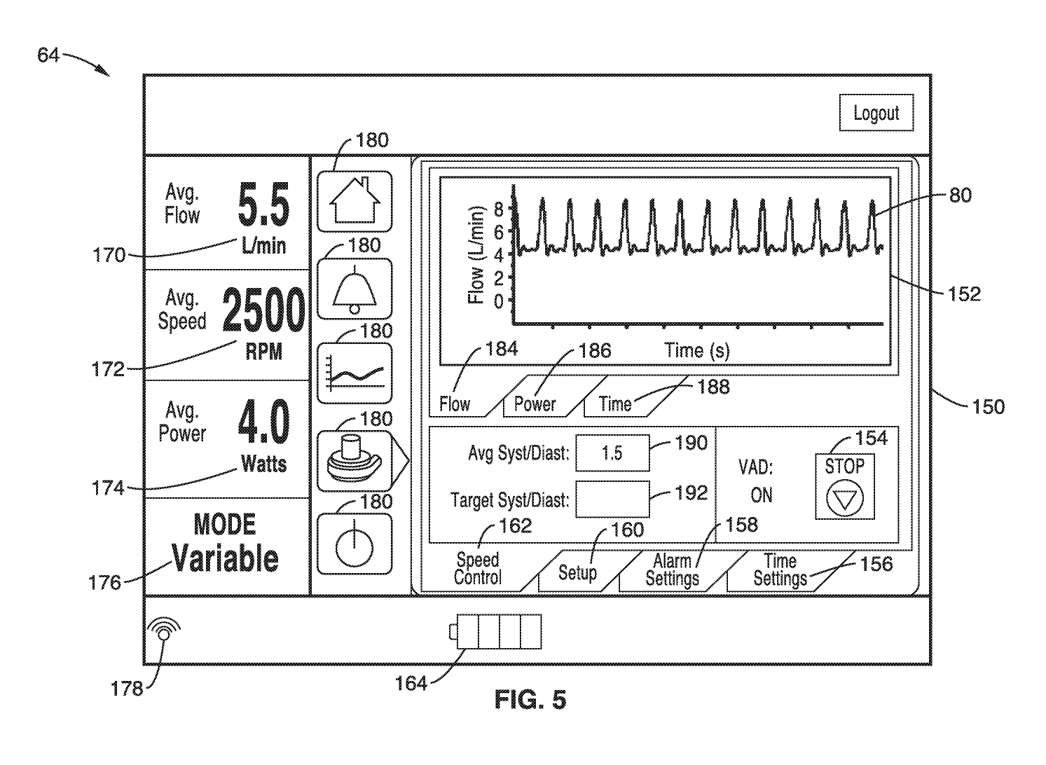

FIG. 5 illustrates an exemplary user interface 64 that may be implemented as application software 56 (FIG. 1). User interface 64 comprises a physician or patient screen 150 that shows pump status in a number of a number of indicators (e.g. average pump flow 170, average pump speed 172, average power 174, and operating mode 176 (e.g. a variable speed mode where the speed of the motor 22 is a varied according to an intra-beat control scheme as detailed in FIG. 3, fixed or constant speed mode, etc.)). A number of graphical buttons 180 may be provided for various modules that are available to the physician and/or patient. A live plot 152 may be output, e.g. showing pump or physiology characteristics such as the flow pulsatility 80.

Each module may have a plurality of tabs 184-188. For example, flow tab 184 may include a field 192 for inputting flow characteristics e.g. target V_{D}/Vₛ compared to the measured or present ratio 190. Power tab 186 may be toggled to adjust the work performed by the pump during the cardiac cycle (in units of watts). Time tab 188 may be toggled to set the ratio of time spent pumping during diastole and systole.

Additional tabs may be provided for time settings 156, alarm settings 158 for events such as high or low current, low flow, suck down, setup 160 and speed control 162. For example, time settings tab 156 may be employed for setting timing for particular intermittent workouts (e.g. for weaning protocols) or duration of assistance or timing of assistance for different phases of recovery (e.g. maximum ventricular unloading for the acute recover phase). Setup tab 160 may provide the physician with desired sensor/feedback input (e.g. pressure, ECG, current sensing, etc.), sensitivity.

Display 152 may also provide a flow pulsatility modification screen similar to that shown in FIG. 2B. In such configuration, the pulsatility curve 80 that has selectable points in which the physician may simply drag on the screen to shape the curve 80 to a desired profile. For example, the physician could drag the systole peak from 90a to 90b (showing a desired target peak from line 82 to 86), or drag a diastole point from 92a to 92b (e.g. from just above trough 84 to line 88. The algorithm 100 would then use that profile as the target profile at which the speed command 44 and timing is set.

The user interface 64 may also include indicators showing various status characteristics of the pump 20. For example, a battery indicator 164 may provide the status of the battery 24 (e.g. percentage of life) and wireless connectivity status indicator 178 may show wireless connection strength with the pump 20.

The systems and methods described above for DVU would provide optimized performance of LVAD's to improve the clinical course of LVAD patients and to improve their quality of life. The following benefits to patients are anticipated: 1) increased rate of weaning from LVAD support and decreased exposure time to the risk of adverse events secondary to the device, 2) improved washing around the apex tube due to smaller EDV and higher flow rates during diastole would decrease the incidence of thrombus formation, 3) greater control over the opening of the aortic valve to maximize washing of the leaflets, 4) increased diastolic augmentation of blood to the heart, and 5) greater durability of weaning.

As an alternative to the above systems and methods, a pump may be configured or programmed to operate under passive, open-loop control. Typically, rotary LVADs use active speed control with a closed-loop control. However, it is possible to run open loop or with simple voltage control. With an open-loop pump under this embodiment, the speed will vary depending upon the load, i.e. the larger the load (e.g. flow) will slow the pump down and the lesser load, the pump will speed up. With respect to rotary pump behavior, if the outlet is clamped (e.g. delta pressure increased) the power drops, so the pump speeds up and pumps more flow up to the current limit. This would be indicative of operation of the pump during diastole. If there is very small delta pressure (as in systole), the pump will not be able to maintain speed because of increased volume presented to the pump, and the current limit will slow down pump, resulting in lower flow. The net effect is that with an open-loop control scenario as described in this embodiment, the ratio of the volume pumped during diastole will be greater than with closed loop speed control, thus passively creating a more favorable pumping scheme than with a pump having closed-loop control and constant speed.

Embodiments of the present technology may be described herein with reference to flowchart illustrations of methods and systems according to embodiments of the technology, and/or procedures, algorithms, steps, operations, formulae, or other computational depictions, which may also be implemented as computer program products. In this regard, each block or step of a flowchart, and combinations of blocks (and/or steps) in a flowchart, as well as any procedure, algorithm, step, operation, formula, or computational depiction can be implemented by various means, such as hardware, firmware, and/or software including one or more computer program instructions embodied in computer-readable program code. As will be appreciated, any such computer program instructions may be executed by one or more computer processors, including without limitation a general purpose computer or special purpose computer, or other programmable processing apparatus to produce a machine, such that the computer program instructions which execute on the computer processor(s) or other programmable processing apparatus create means for implementing the function(s) specified.

Accordingly, blocks of the flowcharts, and procedures, algorithms, steps, operations, formulae, or computational depictions described herein support combinations of means for performing the specified function(s), combinations of steps for performing the specified function(s), and computer program instructions, such as embodied in computer-readable program code logic means, for performing the specified function(s). It will also be understood that each block of the flowchart illustrations, as well as any procedures, algorithms, steps, operations, formulae, or computational depictions and combinations thereof described herein, can be implemented by special purpose hardware-based computer systems which perform the specified function(s) or step(s), or combinations of special purpose hardware and computer-readable program code.

Furthermore, these computer program instructions, such as embodied in computer-readable program code, may also be stored in one or more computer-readable memory or memory devices that can direct a computer processor or other programmable processing apparatus to function in a particular manner, such that the instructions stored in the computer-readable memory or memory devices produce an article of manufacture including instruction means which implement the function specified in the block(s) of the flowchart(s). The computer program instructions may also be executed by a computer processor or other programmable processing apparatus to cause a series of operational steps to be performed on the computer processor or other programmable processing apparatus to produce a computer-implemented process such that the instructions which execute on the computer processor or other programmable processing apparatus provide steps for implementing the functions specified in the block(s) of the flowchart(s), procedure (s) algorithm(s), step(s), operation(s), formula(e), or computational depiction(s).

It will further be appreciated that the terms “programming” or “program executable” as used herein refer to one or more instructions that can be executed by one or more computer processors to perform one or more functions as described herein. The instructions can be embodied in software, in firmware, or in a combination of software and firmware. The instructions can be stored local to the device in non-transitory media, or can be stored remotely such as on a server, or all or a portion of the instructions can be stored locally and remotely. Instructions stored remotely can be downloaded (pushed) to the device by user initiation, or automatically based on one or more factors.

It will further be appreciated that as used herein, that the terms processor, hardware processor, computer processor, central processing unit (CPU), and computer are used synonymously to denote a device capable of executing the instructions and communicating with input/output interfaces and/or peripheral devices, and that the terms processor, hardware processor, computer processor, CPU, and computer are intended to encompass single or multiple devices, single core and multicore devices, and variations thereof.

From the description herein, it will be appreciated that the present disclosure encompasses multiple embodiments which include, but are not limited to, the following:

1. An apparatus for providing mechanical circulatory assistance provided by an implanted left ventricular assist device (LVAD) pump, the apparatus comprising: (a) a processor; and (b) a non-transitory memory storing instructions executable by the processor; (c) wherein said instructions, when executed by the processor, perform steps comprising: (i) receiving data relating to a physiological parameter of a circulatory system in which the implanted LVAD pump is operating; (ii) identifying diastolic and systolic phases of the cardiac cycle of the circulatory system; and (iii) controlling the speed of the pump to have a different speed during the diastolic phase than during the systolic phase.

2. The apparatus, method, or system of any preceding or following embodiment, wherein controlling the speed of the pump comprises increasing the speed of the pump during the diastolic phase as compared to the systolic phase.

3. The apparatus, method, or system of any preceding or following embodiment: wherein said increased speed during the diastolic phase results in an increased pumped diastolic volume; and wherein said increased pumped diastolic volume contributes to a smaller end diastolic volume and a diminished starling response associated with a left ventricle of the circulatory system.

4. The apparatus, method, or system of any preceding or following embodiment, wherein controlling the speed of the pump comprises decreasing the speed of the pump during the systolic phase as compared to the diastolic phase.

5. The apparatus, method, or system of any preceding or following embodiment: wherein said decreased speed of the pump during the systolic phase results in an increased pumped diastolic volume; and wherein said increased pumped systolic volume contributes to a decrease in pressure in an aorta associated with the circulatory system.

6. The apparatus, method, or system of any preceding or following embodiment, wherein controlling the speed of the pump comprises applying a different speed during the diastolic phase than during the systolic phase for specified intervals that are spaced by periods of rest.

7. The apparatus, method, or system of any preceding or following embodiment, wherein the specified intervals comprise periods of one or more of decreasing speed during the diastolic phase and increasing speed in the systolic phase to incrementally reduce assistance provided to the circulatory system by the pump.

8. The apparatus, method, or system of any preceding or following embodiment: wherein receiving data relating to a physiological parameter comprises measuring a current applied to the pump; wherein said current is correlated to an output of the pump and the diastolic and systolic phases of the cardiac cycle; and wherein said instructions when executed by the processor further perform steps comprising identifying the diastolic and systolic phases of the cardiac cycle as a function of the measured current over time.

9. The apparatus, method, or system of any preceding or following embodiment: wherein receiving data relating to a physiological parameter comprises measuring a physiological parameter with a sensor; wherein said physiological parameter is correlated to an output of the pump and the diastolic and systolic phases of the cardiac cycle; and wherein said instructions when executed by the processor further perform steps comprising identifying the diastolic and systolic phases of the cardiac cycle as a function of the measured physiological parameter.

10. The apparatus, method, or system of any preceding or following embodiment, wherein the physiological parameter comprises one or more of electrocardiogram measurements, ventricular pressure measurements or ventricular volume measurements.

11. A method for providing mechanical circulatory assistance provided by a left ventricular assist device (LVAD) pump, the method comprising: installing an LVAD within a circulatory system of a patient; receiving data relating to a physiological parameter of the circulatory system; identifying diastolic and systolic phases of the cardiac cycle of the circulatory system; and controlling the speed of the pump to have a different speed during the diastolic phase than during the systolic phase.

12. The apparatus, method, or system of any preceding or following embodiment, wherein controlling the speed of the pump comprises increasing the speed of the pump during the diastolic phase as compared to the systolic phase.

13. The apparatus, method, or system of any preceding or following embodiment: wherein said increased speed during the diastolic phase results in an increased pumped diastolic volume; and wherein said increased pumped diastolic volume contributes to a smaller end diastolic volume and a diminished starling response associated with a left ventricle of the circulatory system.

14. The apparatus, method, or system of any preceding or following embodiment, wherein controlling the speed of the pump comprises decreasing the speed of the pump during the systolic phase as compared to the diastolic phase.

15. The apparatus, method, or system of any preceding or following embodiment: wherein said decreased speed of the pump during the systolic phase results in a decreased pumped diastolic volume; and wherein said decreased pumped systolic volume contributes to a decrease in pressure in an aorta associated with the circulatory system.

16. The apparatus, method, or system of any preceding or following embodiment, wherein controlling the speed of the pump comprises applying a different speed during the diastolic phase than during the systolic phase for specified intervals that are spaced by periods of rest.

17. The apparatus, method, or system of any preceding or following embodiment, wherein the specified intervals comprise periods of one or more of decreasing speed during the diastolic phase and increasing speed in the systolic phase to incrementally reduce assistance provided to the circulatory system by the pump.

18. The apparatus, method, or system of any preceding or following embodiment: wherein receiving data relating to a physiological parameter comprises measuring a current applied to the pump; wherein said current is correlated to an output of the pump and the diastolic and systolic phases of the cardiac cycle; and wherein the diastolic and systolic phases of the cardiac cycle are identified as a function of the measured current over time.

19. The apparatus, method, or system of any preceding or following embodiment: wherein receiving data relating to a physiological parameter comprises measuring a physiological parameter with a sensor; wherein said physiological parameter is correlated to an output of the pump and the diastolic and systolic phases of the cardiac cycle; and wherein the diastolic and systolic phases of the cardiac cycle are identified as a function of the measured physiological parameter.

20. The apparatus, method, or system of any preceding or following embodiment, wherein the physiological parameter comprises one or more of electrocardiogram measurements, ventricular pressure measurements or ventricular volume measurements.

21. A system for providing mechanical circulatory assistance to a patient, the system comprising: (a) a left ventricular assist device (LVAD) pump configured to be inserted into the patient's circulatory system; (b) a processor; and (c) a non-transitory memory storing instructions executable by the processor; (d) wherein said instructions, when executed by the processor, perform steps comprising: (i) receiving data relating to a physiological parameter of the circulatory system; (ii) identifying diastolic and systolic phases of the cardiac cycle of the circulatory system; and (iii) controlling the speed of the pump to have a different speed during the diastolic phase than during the systolic phase.

22. The apparatus, method, or system of any preceding or following embodiment, wherein controlling the speed of the pump comprises increasing the speed of the pump during the diastolic phase as compared to the systolic phase.

23. The apparatus, method, or system of any preceding or following embodiment: wherein said increased speed during the diastolic phase results in an increased pumped diastolic volume; and wherein said increased pumped diastolic volume contributes to a smaller end diastolic volume and a diminished starling response associated with a left ventricle of the circulatory system.

24. The apparatus, method, or system of any preceding or following embodiment, wherein controlling the speed of the pump comprises decreasing the speed of the pump during the systolic phase as compared to the diastolic phase.

25. The apparatus, method, or system of any preceding or following embodiment: wherein said decreased speed of the pump during the systolic phase results in an decreased pumped diastolic volume; and wherein said decrease pumped systolic volume contributes to a decrease in pressure in an aorta associated with the circulatory system.

26. The apparatus, method, or system of any preceding or following embodiment, wherein controlling the speed of the pump comprises applying a different speed during the diastolic phase than during the systolic phase for specified intervals that are spaced by periods of rest.

27. The apparatus, method, or system of any preceding or following embodiment, wherein the specified intervals comprise periods of one or more of decreasing speed during the diastolic phase and increasing speed in the systolic phase to incrementally reduce assistance provided to the circulatory system by the pump.

28. The apparatus, method, or system of any preceding or following embodiment: wherein receiving data relating to a physiological parameter comprises measuring a current applied to the pump; wherein said current is correlated to an output of the pump and the diastolic and systolic phases of the cardiac cycle; wherein said instructions when executed by the processor further perform steps comprising identifying the diastolic and systolic phases of the cardiac cycle as a function of the measured current over time.

29. The apparatus, method, or system of any preceding or following embodiment, further comprising: one or more sensors coupled to the processor; wherein receiving data relating to a physiological parameter comprises measuring a physiological parameter with the one or more sensors; wherein said physiological parameter is correlated to an output of the pump and the diastolic and systolic phases of the cardiac cycle; and wherein said instructions when executed by the processor further perform steps comprising identifying the diastolic and systolic phases of the cardiac cycle as a function of the measured physiological parameter.

30. The apparatus, method, or system of any preceding or following embodiment, wherein the physiological parameter comprises one or more of electrocardiogram measurements, ventricular pressure measurements or ventricular volume measurements.

31. The apparatus, method, or system of any preceding or following embodiment, further comprising: an external device coupled on the pump; wherein the external device comprises said instructions to remotely control the speed of the pump from a location external to the patient.

32. The apparatus, method, or system of any preceding or following embodiment: wherein said instructions are configured to allow user input of a target physiological metric; and wherein controlling the speed of the pump comprises: (i) calculating a change in pump speed for one or more of the diastolic phase and the systolic phase based on the target physiological metric; and (ii) sending a command to the pump to change the pump speed at a specified period timed according to one or more of the diastolic phase and the systolic phase.

33. The apparatus, method, or system of any preceding or following embodiment, wherein said instructions are further configured to perform the steps of: (iv) receiving data relating to an adjusted physiological parameter of the circulatory system resulting from the change in pump speed; (v) comparing the target physiological metric to the adjusted physiological parameter; and (vi) calculating an adjusted change in pump speed in response to the target physiological metric not being met.

34. The apparatus, method, or system of any preceding or following embodiment, wherein the target physiological metric comprises a ratio of diastolic volume to systolic volume.

35. The apparatus, method, or system of any preceding or following embodiment, wherein the external device comprises a user interface configured to allow user input of the target physiological metric.

As used herein, the singular terms “a,” “an,” and “the” may include plural referents unless the context clearly dictates otherwise. Reference to an object in the singular is not intended to mean “one and only one” unless explicitly so stated, but rather “one or more.”

As used herein, the term “set” refers to a collection of one or more objects. Thus, for example, a set of objects can include a single object or multiple objects.

As used herein, the terms “substantially” and “about” are used to describe and account for small variations. When used in conjunction with an event or circumstance, the terms can refer to instances in which the event or circumstance occurs precisely as well as instances in which the event or circumstance occurs to a close approximation. When used in conjunction with a numerical value, the terms can refer to a range of variation of less than or equal to ±10% of that numerical value, such as less than or equal to ±5%, less than or equal to ±4%, less than or equal to ±3%, less than or equal to ±2%, less than or equal to ±1%, less than or equal to ±0.5%, less than or equal to ±0.1%, or less than or equal to ±0.05%. For example, “substantially” aligned can refer to a range of angular variation of less than or equal to ±10°, such as less than or equal to ±5°, less than or equal to ±4°, less than or equal to ±3°, less than or equal to ±2°, less than or equal to ±1°, less than or equal to ±0.5°, less than or equal to ±0.1°, or less than or equal to ±0.05°.

Additionally, amounts, ratios, and other numerical values may sometimes be presented herein in a range format. It is to be understood that such range format is used for convenience and brevity and should be understood flexibly to include numerical values explicitly specified as limits of a range, but also to include all individual numerical values or sub-ranges encompassed within that range as if each numerical value and sub-range is explicitly specified. For example, a ratio in the range of about 1 to about 200 should be understood to include the explicitly recited limits of about 1 and about 200, but also to include individual ratios such as about 2, about 3, and about 4, and sub-ranges such as about 10 to about 50, about 20 to about 100, and so forth.

Although the description herein contains many details, these should not be construed as limiting the scope of the disclosure but as merely providing illustrations of some of the presently preferred embodiments. Therefore, it will be appreciated that the scope of the disclosure fully encompasses other embodiments which may become obvious to those skilled in the art.

All structural and functional equivalents to the elements of the disclosed embodiments that are known to those of ordinary skill in the art are expressly incorporated herein by reference and are intended to be encompassed by the present claims. Furthermore, no element, component, or method step in the present disclosure is intended to be dedicated to the public regardless of whether the element, component, or method step is explicitly recited in the claims. No claim element herein is to be construed as a “means plus function” element unless the element is expressly recited using the phrase “means for”. No claim element herein is to be construed as a “step plus function” element unless the element is expressly recited using the phrase “step for”.

What is claimed is:

1. An apparatus for providing mechanical circulatory assistance provided by an implanted left ventricular assist device (LVAD) pump, the apparatus comprising:
(a) a processor; and
(b) a non-transitory memory storing instructions executable by the processor;
(c) wherein said instructions, when executed by the processor, perform steps comprising:
(i) receiving data relating to a physiological parameter of a circulatory system in which the implanted LVAD pump is operating;
(ii) identifying diastolic and systolic phases of the cardiac cycle of the circulatory system; and
(iii) controlling the speed of the pump to have a different speed during the diastolic phase than during the systolic phase.

2. The apparatus of claim 1, wherein controlling the speed of the pump comprises increasing the speed of the pump during the diastolic phase as compared to the systolic phase.

3. The apparatus of claim 2:
wherein said increased speed during the diastolic phase results in an increased pumped diastolic volume; and
wherein said increased pumped diastolic volume contributes to a smaller end diastolic volume and a diminished starling response associated with a left ventricle of the circulatory system.

4. The apparatus of claim 1, wherein controlling the speed of the pump comprises decreasing the speed of the pump during the systolic phase as compared to the diastolic phase.

5. The apparatus of claim 4:
wherein said decreased speed of the pump during the systolic phase results in an increased pumped diastolic volume; and
wherein said increased pumped systolic volume contributes to a decrease in pressure in an aorta associated with the circulatory system.

6. The apparatus of claim 1, wherein controlling the speed of the pump comprises applying a different speed during the diastolic phase than during the systolic phase for specified intervals that are spaced by periods of rest.

7. The apparatus of claim 6, wherein the specified intervals comprise periods of one or more of decreasing speed during the diastolic phase and increasing speed in the systolic phase to incrementally reduce assistance provided to the circulatory system by the pump.

8. The apparatus of claim 1:
wherein receiving data relating to a physiological parameter comprises measuring a current applied to the pump;
wherein said current is correlated to an output of the pump and the diastolic and systolic phases of the cardiac cycle; and
wherein said instructions when executed by the processor further perform steps comprising identifying the diastolic and systolic phases of the cardiac cycle as a function of the measured current over time.

9. The apparatus of claim 1:
wherein receiving data relating to a physiological parameter comprises measuring a physiological parameter with a sensor;
wherein said physiological parameter is correlated to an output of the pump and the diastolic and systolic phases of the cardiac cycle; and
wherein said instructions when executed by the processor further perform steps comprising identifying the diastolic and systolic phases of the cardiac cycle as a function of the measured physiological parameter.

10. The apparatus of claim 9, wherein the physiological parameter comprises one or more of electrocardiogram measurements, ventricular pressure measurements or ventricular volume measurements.

11. A method for providing mechanical circulatory assistance provided by a left ventricular assist device (LVAD) pump, the method comprising:
installing an LVAD within a circulatory system of a patient;
receiving data relating to a physiological parameter of the circulatory system;
identifying diastolic and systolic phases of the cardiac cycle of the circulatory system; and
controlling the speed of the pump to have a different speed during the diastolic phase than during the systolic phase.

12. The method of claim 11, wherein controlling the speed of the pump comprises increasing the speed of the pump during the diastolic phase as compared to the systolic phase.

13. The method of claim 12:
wherein said increased speed during the diastolic phase results in an increased pumped diastolic volume; and
wherein said increased pumped diastolic volume contributes to a smaller end diastolic volume and a diminished starling response associated with a left ventricle of the circulatory system.

14. The method of claim 11, wherein controlling the speed of the pump comprises decreasing the speed of the pump during the systolic phase as compared to the diastolic phase.

15. The method of claim 14:
wherein said decreased speed of the pump during the systolic phase results in a decreased pumped diastolic volume; and
wherein said decreased pumped systolic volume contributes to a decrease in pressure in an aorta associated with the circulatory system.

16. The method of claim 11, wherein controlling the speed of the pump comprises applying a different speed during the diastolic phase than during the systolic phase for specified intervals that are spaced by periods of rest.

17. The method of claim 17, wherein the specified intervals comprise periods of one or more of decreasing speed during the diastolic phase and increasing speed in the systolic phase to incrementally reduce assistance provided to the circulatory system by the pump.

18. The method of claim 11:
wherein receiving data relating to a physiological parameter comprises measuring a current applied to the pump;
wherein said current is correlated to an output of the pump and the diastolic and systolic phases of the cardiac cycle; and
wherein said method further comprises identifying the diastolic and systolic phases of the cardiac cycle as a function of the measured current over time.

19. The method of claim 11:
wherein receiving data relating to a physiological parameter comprises measuring a physiological parameter with a sensor;
wherein said physiological parameter is correlated to an output of the pump and the diastolic and systolic phases of the cardiac cycle; and
wherein said method further comprises identifying the diastolic and systolic phases of the cardiac cycle as a function of the measured physiological parameter.

20. The method of claim 19, wherein the physiological parameter comprises one or more of electrocardiogram measurements, ventricular pressure measurements or ventricular volume measurements.

21. A system for providing mechanical circulatory assistance to a patient, the system comprising:
(a) a left ventricular assist device (LVAD) pump configured to be inserted into the patient's circulatory system;
(b) a processor; and
(c) a non-transitory memory storing instructions executable by the processor;
(d) wherein said instructions, when executed by the processor, perform steps comprising:
(i) receiving data relating to a physiological parameter of the circulatory system;
(ii) identifying diastolic and systolic phases of the cardiac cycle of the circulatory system; and
(iii) controlling the speed of the pump to have a different speed during the diastolic phase than during the systolic phase.

22. The system of claim 21, wherein controlling the speed of the pump comprises increasing the speed of the pump during the diastolic phase as compared to the systolic phase.

23. The system apparatus of claim 22:
wherein said increased speed during the diastolic phase results in an increased pumped diastolic volume; and
wherein said increased pumped diastolic volume contributes to a smaller end diastolic volume and a diminished starling response associated with a left ventricle of the circulatory system.

24. The system of claim 21, wherein controlling the speed of the pump comprises decreasing the speed of the pump during the systolic phase as compared to the diastolic phase.

25. The system of claim 24:
wherein said decreased speed of the pump during the systolic phase results in an decreased pumped diastolic volume; and
wherein said decrease pumped systolic volume contributes to a decrease in pressure in an aorta associated with the circulatory system.

26. The system of claim 21, wherein controlling the speed of the pump comprises applying a different speed during the diastolic phase than during the systolic phase for specified intervals that are spaced by periods of rest.

27. The system of claim 26, wherein the specified intervals comprise periods of one or more of decreasing speed during the diastolic phase and increasing speed in the systolic phase to incrementally reduce assistance provided to the circulatory system by the pump.

28. The system of claim 21:
wherein receiving data relating to a physiological parameter comprises measuring a current applied to the pump;
wherein said current is correlated to an output of the pump and the diastolic and systolic phases of the cardiac cycle;
wherein said instructions when executed by the processor further perform steps comprising identifying the diastolic and systolic phases of the cardiac cycle as a function of the measured current over time.

29. The system of claim 21, further comprising:
one or more sensors coupled to the processor;
wherein receiving data relating to a physiological parameter comprises measuring a physiological parameter with the one or more sensors;
wherein said physiological parameter is correlated to an output of the pump and the diastolic and systolic phases of the cardiac cycle; and
wherein said instructions when executed by the processor further perform steps comprising identifying the diastolic and systolic phases of the cardiac cycle as a function of the measured physiological parameter.

30. The system of claim 29, wherein the physiological parameter comprises one or more of electrocardiogram measurements, ventricular pressure measurements or ventricular volume measurements.

31. The system of claim 21, further comprising:
an external device coupled on the pump;
wherein the external device comprises said instructions to remotely control the speed of the pump from a location external to the patient.

32. The system of claim 31:
wherein said instructions are configured to allow user input of a target physiological metric; and
wherein controlling the speed of the pump comprises:
(i) calculating a change in pump speed for one or more of the diastolic phase and the systolic phase based on the target physiological metric; and
(ii) sending a command to the pump to change the pump speed at a specified period timed according to one or more of the diastolic phase and the systolic phase.

33. The system of claim 32, wherein said instructions are further configured to perform the steps of:
(iv) receiving data relating to an adjusted physiological parameter of the circulatory system resulting from the change in pump speed;
(v) comparing the target physiological metric to the adjusted physiological parameter; and
(vi) calculating an adjusted change in pump speed in response to the target physiological metric not being met.

34. The system of claim 31, wherein the target physiological metric comprises a ratio of diastolic volume to systolic volume.

35. The system of claim 31, wherein the external device comprises a user interface configured to allow user input of the target physiological metric.
